(19) United States
(12) Patent Application Publication
(10) Pub. No.: US 2025/0380737 A1
(43) Pub. Date: Dec. 18, 2025

(54) INHALER PROVIDING VIBRATION FOR POWDER INHALATION

(71) Applicant: KT&G CORPORATION, Daejeon (KR)

(72) Inventors: Min Kyu KIM, Seoul (KR); Paul Joon SUNWOO, Seoul (KR); Won Kyeong LEE, Guri-si (KR)

(73) Assignee: KT&G CORPORATION, Daejeon (KR)

(21) Appl. No.: 18/876,036

(22) PCT Filed: Sep. 1, 2023
(86) PCT No.: PCT/KR2023/013065

§ 371 Date: Dec. 17, 2024

(30) Foreign Application Priority Data
Sep. 6, 2022 (KR) 10-2022-0112846
Dec. 13, 2022 (KR) 10-2022-0173871

(51) Int. Cl. A24F 40/20; A24F 40/05; A24F 40/51

(52) U.S. Cl. CPC A24F 40/20; A24F 40/05; A24F 40/51

(57) ABSTRACT

An inhaler includes a stick provided with a chamber configured to accommodate a capsule comprising powder and a piercing hole open toward the chamber, a holder including an insertion groove into which the stick is inserted in a first direction, a piercing member provided in the insertion groove and configured to crush a capsule by passing through the piercing hole when the stick is inserted into the insertion groove, a vibration member configured to provide a vibration to the piercing member, a puff sensor configured to sense airflow inside the stick, and a controller configured to control an operation of the inhaler.

### TECHNICAL FIELD

Various embodiments of the present disclosure relate to an inhaler.

### BACKGROUND ART

Recently, demands for alternative articles to overcome disadvantages of general cigarettes have increased. For example, an inhaler is a device for a user to inhale a liquid or gas including a composition such as a drug through the oral cavity or nasal cavity.

Such devices may have a chamber containing an inhalable composition, and the composition may pass from the chamber through a channel and finally to the oral cavity or nasal cavity to be inhaled by the user.

The above description has been possessed or acquired by the inventor(s) in the course of conceiving the present disclosure and is not necessarily an art publicly known before the present application is filed.

### DISCLOSURE OF THE INVENTION

### Technical Problem

In an inhaler using a composition in a powder state, a conventional non-electronic inhaler has to inhale powder depending on a user's breathing. In this case, the amount of powder discharged from the inhaler may vary according to the user's pulmonary respiration volume, and a user whose pulmonary respiration volume does not meet a determined level has a problem in that the use of the inhaler is restricted.

In order to solve this problem, there has been a demand for an inhaler with which even a user with a weak pulmonary respiration may smoothly inhale and furthermore, which may allow a general user to individually control the discharge state of the powder.

### Solution to Problem

An inhaler according to an embodiment includes a stick including a chamber configured to accommodate a capsule including powder and a piercing hole open toward the chamber, a holder including an insertion groove into which the stick is inserted in a first direction, a piercing member provided in the insertion groove and configured to crush a capsule by passing through the piercing hole when the stick is inserted into the insertion groove, a vibration member configured to provide a vibration to the piercing member, a puff sensor configured to sense airflow inside the stick, and a controller configured to control an operation of the inhaler.

The controller may be configured to adjust an intensity of a vibration of the vibration member based on a value of an inhalation pressure sensed by the puff sensor.

The controller may be configured to control the vibration member so that a preset first vibration intensity appears when the value of the inhalation pressure corresponds to a preset first threshold value.

The controller may be configured to stop the vibration of the vibration member when the value of the inhalation pressure exceeds a preset second threshold value.

The controller may be configured to control the vibration member so that a vibration intensity inversely proportional to the value of the inhalation pressure appears when the value of the inhalation pressure is greater than the preset first threshold value and equal to or less than the preset second threshold value.

The preset first threshold value may be a personalized value for a user of the inhaler.

The inhaler may further include a sub-vibration member configured to provide a vibration to the chamber.

The inhaler may further include an elastic member provided in the insertion groove and pressed by the stick when the stick is inserted, wherein the sub-vibration member may be configured to provide the vibration to the chamber of the stick through the elastic member.

The sub-vibration member may be configured to vibrate the chamber in a direction substantially parallel to the first direction.

The sub-vibration member may be configured to vibrate the chamber in a direction substantially perpendicular to the first direction.

The stick may include a mouthpiece provided on an opposite side of the chamber, an airflow channel configured to communicate from the chamber to the mouthpiece, and a mesh arranged between the airflow channel and the chamber.

The stick may further include a sealing member configured to seal the piercing hole and crushed by the piercing member when the stick is inserted into the insertion groove.

A method of providing a vibration for powder inhalation, the method being performed by an inhaler, includes controlling the vibration member so that a preset first vibration intensity appears when a first value of an inhalation pressure measured by the puff sensor corresponds to a preset first threshold value and controlling the vibration member so that a second vibration intensity inversely proportional to a second value of the inhalation pressure appears when the second value is equal to or greater than the preset first threshold value, wherein the inhaler includes a stick provided with a chamber configured to accommodate a capsule including powder and a piercing hole open toward the chamber, a holder including an insertion groove into which the stick is inserted in a first direction, a piercing member provided in the insertion groove and configured to crush the capsule by passing through the piercing hole when the stick is inserted into the insertion groove, a vibration member configured to provide a vibration to the piercing member, a puff sensor configured to sense airflow inside the stick, and a controller configured to control an operation of the inhaler.

The method may further include stopping the vibration of the vibration member when a third value of the inhalation pressure exceeds a preset second threshold value.

The method may further include stopping the vibration of the vibration member when a fourth value of the inhalation pressure is less than the preset first threshold value.

### Advantageous Effects of Invention

According to an embodiment, an inhaler may provide a vibration for powder inhalation.

The effects of the inhaler are not limited to the above-mentioned effects, and other unmentioned effects can be clearly understood from the following description by one of ordinary skill in the art to which the present disclosure pertains.

### BRIEF DESCRIPTION OF DRAWINGS

FIG. 1 is a block diagram illustrating an inhaler according to an embodiment.

FIG. 2 is a diagram schematically illustrating an inhaler according to an embodiment.

FIG. 3A is a diagram schematically illustrating the inside of an inhaler according to an embodiment.

FIG. 3B is a diagram schematically illustrating the inside of an inhaler according to an embodiment.

FIG. 4A is a diagram schematically illustrating the inside of an inhaler according to an embodiment.

FIG. 4B is a diagram schematically illustrating the inside of an inhaler according to an embodiment.

FIG. 5A is a cross-sectional view of a stick according to an embodiment.

FIG. 5B is a cross-sectional view of a stick according to an embodiment.

FIG. 6 is a flowchart illustrating a method of controlling a vibration of a vibration member, according to an embodiment.

FIG. 7 illustrates a vibration intensity generated according to an inhalation pressure, according to an embodiment.

### BEST MODE FOR CARRYING OUT THE INVENTION

The terms used in various embodiments are selected from among common terms that are currently widely used, in consideration of their function in the disclosure. However, the terms may become different according to an intention of one of ordinary skill in the art, a precedent, or the advent of new technology. Also, in particular cases, the terms are discretionally selected by the applicant of the disclosure, and the meaning of those terms will be described in detail in the corresponding part of the detailed description. Therefore, the terms used in the disclosure are not merely designations of the terms, but the terms are defined based on the meaning of the terms and content throughout the disclosure.

It will be understood that when a certain part “includes” a certain component, the part does not exclude another component but may further include another component, unless the context clearly dictates otherwise. Also, terms such as “unit,” “module,” etc., as used in the specification may refer to a part for processing at least one function or operation and which may be implemented as hardware, software, or a combination of hardware and software.

As used herein, an expression such as “at least one of” that precedes listed components modifies not each of the listed components but all the listed components. For example, expressions “at least one of a, b, or c” and “at least one of a, b, and c” should be construed as including a, b, c, a and b, a and c, b and c, or a, b, and c.

In various embodiments, the term “puff” refers to inhalation by a user, and inhalation refers to a situation in which a user draws in an aerosol into their oral cavity, nasal cavity, or lungs through the mouth or nose.

In an embodiment, an inhaler may include a main body (or a holder) configured to support a cartridge (or a stick) configured to accommodate a capsule containing a composition. The cartridge may be detachably coupled to the main body. However, embodiments are not limited thereto. The cartridge may be integrally formed or assembled with the main body and may be secured to the main body so as not to be detached by a user. The cartridge may be mounted on the main body while the capsule is accommodated therein. However, embodiments are not limited thereto. For example, powder or a capsule containing the powder may be injected into the cartridge while the cartridge is coupled to the main body.

Hereinafter, embodiments of the disclosure are described in detail with reference to the accompanying drawings such that one of ordinary skill in the art may easily practice the disclosure. The disclosure may be practiced in forms that are implementable in the inhaler according to various embodiments described above or may be embodied and practiced in many different forms and is not limited to the embodiments described herein.

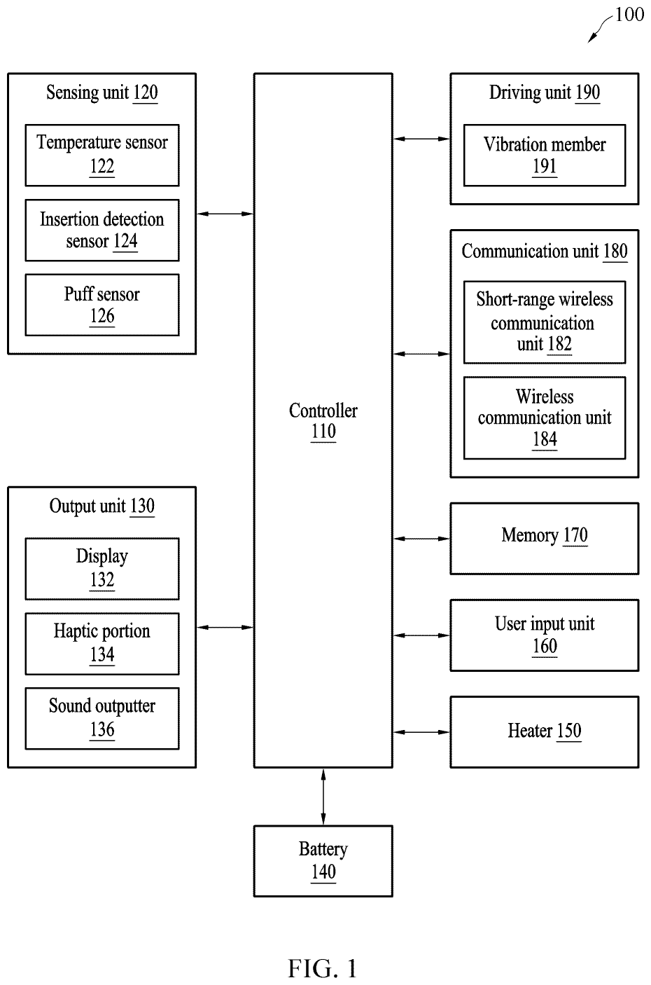

FIG. 1 is a block diagram illustrating an inhaler 100 according to an embodiment.

Referring to FIG. 1, the inhaler 100 may include at least one of a controller 110, a sensing unit 120, an output unit 130, a battery 140, a heater 150, a user input unit 160, a memory 170, a communication unit 180, and a driving unit 190.

However, an internal structure of the inhaler 100 is not limited to that shown in FIG. 1. It is to be understood by one of ordinary skill in the art to which the present disclosure pertains that some of the components shown in FIG. 1 may be omitted or new components may be added according to the design of the inhaler 100.

In an embodiment, the sensing unit 120 may sense a state of the inhaler 100 or a state of an environment around the inhaler 100 and transmit sensed information to the controller 110 (or a processor). The controller 110 may control driving of other components of the inhaler 100 based on the sensed information.

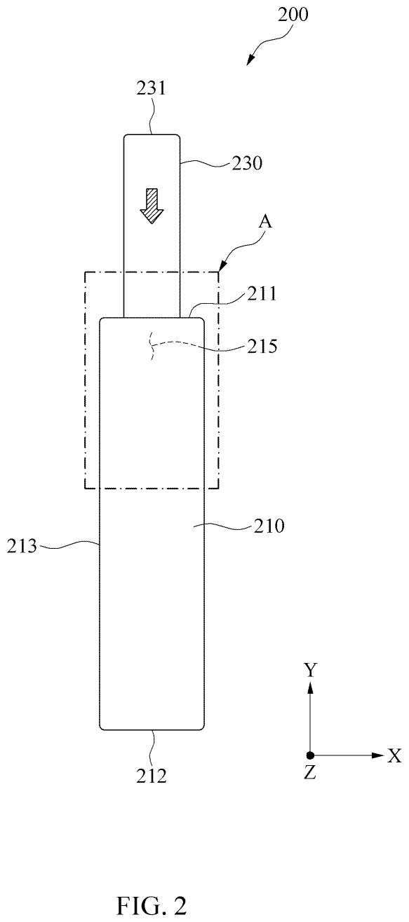

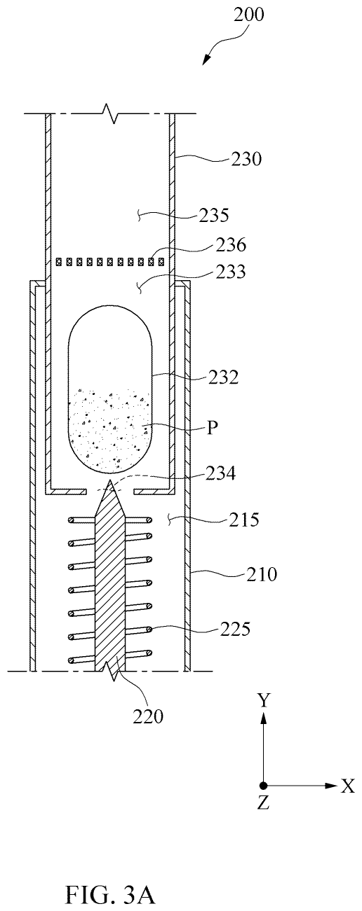

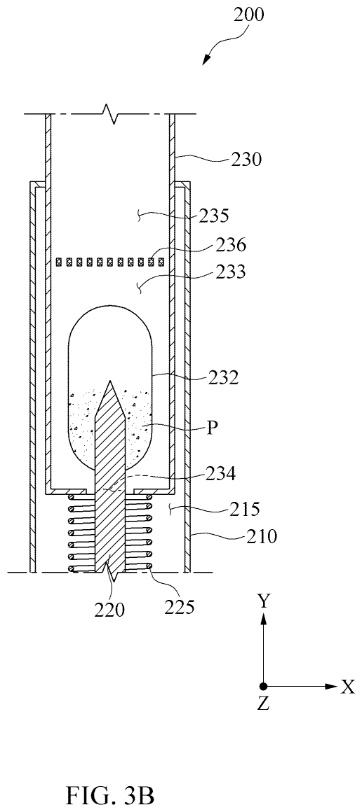

For example, the controller 110 may perform various functions, such as controlling an operation of the heater 150 based on a sensing result of the sensing unit 120, controlling the driving unit 190 by determining whether a stick (e.g., a stick 230 of FIG. 2), a capsule (e.g., a capsule 232 of FIG. 3A), a cartridge, or a cigarette is inserted, displaying a notification on the output unit 130, or the like.

In an embodiment, the sensing unit 120 may include at least one of a temperature sensor 122, an insertion detection sensor 124, or a puff sensor 126. However, embodiments are not limited thereto.

In an embodiment, the temperature sensor 122 may sense a temperature at which the heater 150 is heated. The inhaler 100 may include a separate temperature sensor for sensing the temperature of the heater 150, or the heater 150 itself may perform a function as a temperature sensor. Alternatively, the temperature sensor 122 may be arranged around the battery 140 to monitor a temperature of the battery 140.

In an embodiment, the insertion detection sensor 124 may detect insertion and/or removal of a stick (e.g., the stick 230 of FIG. 2) or a capsule (e.g., the capsule 232 of FIGS. 3A and 3B). The insertion detection sensor 124 may include, for example, at least one of a film sensor, a pressure sensor, a light sensor, a resistive sensor, a capacitive sensor, an inductive sensor, or an infrared sensor, and may sense a signal change by the insertion and/or removal of the stick or capsule.

In an embodiment, the puff sensor 126 may sense a puff by a user based on various physical changes in an airflow path or airflow channel. For example, the puff sensor 126 may sense the puff by the user based on one of a temperature change, a flow change, a voltage change, and a pressure change.

In an embodiment, the sensing unit 120 may further include at least one of a photoplethysmography (PPG) sensor, a temperature/humidity sensor, an atmospheric pressure sensor, a magnetic sensor, an acceleration sensor, a gyroscope sensor, a position sensor (e.g., a global positioning system (GPS)), a proximity sensor, or a red, green, blue (RGB) sensor (e.g., an illuminance sensor), in addition to the sensors described above. A function of each sensor may be intuitively inferable from its name by one of ordinary skill in the art, and thus, a detailed description thereof is omitted herein.

In an embodiment, the output unit 130 may output information about a state of the inhaler 100 and provide the information to the user. The output unit 130 may include at least one of a display 132, a haptic portion 134, or a sound outputter 136. However, embodiments are not limited thereto. When the display 132 and a touchpad are provided in a layered structure to form a touchscreen, the display 132 may be used as an input device in addition to an output device.

In an embodiment, the display 132 may visually provide information about the inhaler 100 to the user. For example, the information about the inhaler 100 may include a variety of information, for example, information about at least one of a charging/discharging state of the battery 140 of the inhaler 100, a preheating state of the heater 150, an insertion/removal state of a stick or a capsule, a state (e.g., an abnormal item detection) in which use of the inhaler 100 is restricted, and a vibration state of the driving unit 190, and the display 132 may externally output the above information. The display 132 may be, for example, a liquid-crystal display (LCD) panel, an organic light-emitting display (OLED) panel, and the like. The display 132 may also be in the form of a light-emitting diode (LED) device.

In an embodiment, the haptic portion 134 may provide information about the inhaler 100 to the user in a haptic way by converting an electrical signal into a mechanical stimulus or an electrical stimulus. The haptic portion 134 may include, for example, a motor, a piezoelectric element, or an electrical stimulation device.

In an embodiment, the sound outputter 136 may provide the information about the inhaler 100 to the user in an auditory way. For example, the sound outputter 136 may convert an electrical signal into a sound signal and externally output the sound signal.

In an embodiment, the battery 140 may supply power to be used to operate the inhaler 100. The battery 140 may supply power to heat the heater 150.

In addition, the battery 140 may supply power required for operations of the other components (e.g., the sensing unit 120, the output unit 130, the user input unit 160, the memory 170, the communication unit 180, or the driving unit 190) included in the inhaler 100. The battery 140 may be a rechargeable battery or a disposable battery. The battery 140 may be, for example, a lithium polymer (LiPoly) battery. However, embodiments are not limited thereto.

In an embodiment, the heater 150 may receive power from the battery 140 to heat an aerosol generating material. Although not shown in FIG. 1, the inhaler 100 may further include a power conversion circuit (e.g., a direct current (DC)-to-DC (DC/DC) converter) that converts power of the battery 140 and supplies the power to the heater 150. When the inhaler 100 generates an aerosol in an induction heating manner, the inhaler 100 may further include a DC-to-alternating current (AC) (DC/AC) converter that converts DC power of the battery 140 into AC power.

In an embodiment, the controller 110, the sensing unit 120, the output unit 130, the user input unit 160, the memory 170, the communication unit 180, and the driving unit 190 may receive power from the battery 140 to perform functions. Although not shown in FIG. 1, the inhaler 100 may further include a power conversion circuit, for example, a low dropout (LDO) circuit or a voltage regulator circuit, which converts power of the battery 140 and supplies the power to respective components.

In an embodiment, the heater 150 may be formed of any suitable electrically resistive material. The electrically resistive material may be, for example, a metal or a metal alloy including titanium, zirconium, tantalum, platinum, nickel, cobalt, chromium, hafnium, niobium, molybdenum, tungsten, tin, gallium, manganese, iron, copper, stainless steel, nichrome, or the like. However, embodiments are not limited thereto. In addition, the heater 150 may be implemented as a metal heating wire, a metal heating plate on which an electrically conductive track is arranged, a ceramic heating element, or the like. However, embodiments are not limited thereto.

According to an embodiment, the heater 150 may be an induction heater. For example, the heater 150 may include a susceptor that heats the aerosol generating material by generating heat through a magnetic field applied by a coil.

In an embodiment, the heater 150 may include a plurality of heaters. For example, the heater 150 may include a first heater for heating an aerosol generating article and a second heater for heating a liquid.

In an embodiment, the user input unit 160 may receive information input from the user or may output information to the user. For example, the user input unit 160 may include a keypad, a dome switch, a touchpad (e.g., a contact capacitive type, a pressure resistive film type, an infrared sensing type, a surface ultrasonic conduction type, an integral tension measurement type, a piezo effect method, etc.), a jog wheel, a jog switch, or the like. However, embodiments are not limited thereto. In addition, although not shown in FIG. 1, the inhaler 100 may further include a connection interface such as a universal serial bus (USB) interface and may be connected to another external device through the connection interface such as a USB interface to transmit and receive information or to charge the battery 140.

In an embodiment, the memory 170, which is hardware for storing various pieces of data processed by the inhaler 100, may store data processed by the controller 110 and data to be processed thereby. The memory 170 may include at least one type of storage medium of flash memory type memory, hard disk type memory, multimedia card micro type memory, card type memory (e.g., SD or xD memory), random-access memory (RAM), static random-access memory (SRAM), read-only memory (ROM), electrically erasable programmable read-only memory (EEPROM), programmable read-only memory (PROM), magnetic memory, a magnetic disk, or an optical disk.

In an embodiment, the memory 170 may store an operating time of the inhaler 100, the maximum number of puffs, the current number of puffs, at least one temperature profile, data associated with a smoking pattern of a user, or the like.

In an embodiment, the communication unit 180 may include at least one component for communicating with another electronic device. For example, the communication unit 180 may include a short-range wireless communication unit 182 and a wireless communication unit 184.

In an embodiment, the short-range wireless communication unit 182 may include a Bluetooth communication unit, a BLE communication unit, a near field communication unit, a wireless local area network (WLAN) communication unit (e.g., Wi-Fi), a ZigBee communication unit, an infrared data association (IrDA) communication unit, a Wi-Fi direct (WFD) communication unit, an ultra-wideband (UWB) communication unit, and an Ant+ communication unit. However, embodiments are not limited thereto.

In an embodiment, the wireless communication unit 184 may include, for example, a cellular network communicator, an Internet communicator, a computer network (e.g., a local area network (LAN) or a wide-area network (WAN)) communicator, or the like. However, embodiments are not limited thereto. The wireless communication unit 184 may use subscriber information (e.g., international mobile subscriber identity (IMSI)) to identify and authenticate the inhaler 100 in a communication network.

In an embodiment, the driving unit 190 may include various driving devices to assist an inhalation motion of a user using the inhaler 100. For example, the driving unit 190 may include a vibration member 191 to assist in delivering powder of the inhaler 100.

In an embodiment, the vibration member 191 may be implemented as an electronic vibrator. When a voltage (e.g., AC voltage) is applied to the vibration member 191, the vibration member 191 may generate a vibration in response to the voltage. However, embodiments are not limited thereto, and the driving unit 190 may further include an element such as, for example, a motor, a shaft, a plurality of pinions, or a hydraulic device.

In an embodiment, the controller 110 may control the overall operation of the inhaler 100. In an embodiment, the controller 110 may include at least one processor. The processor may be implemented as an array of a plurality of logic gates or may be implemented as a combination of a general-purpose microprocessor and a memory in which a program executable by the microprocessor is stored. In addition, it is to be understood by one of ordinary skill in the art to which the present disclosure pertains that the processor may be implemented in other types of hardware.

In an embodiment, the controller 110 may control the temperature of the heater 150 by controlling supply of power from the battery 140 to the heater 150. For example, the controller 110 may control the supply of power by controlling switching of a switching element between the battery 140 and the heater 150. In another example, a direct heating circuit may control the supply of power to the heater 150 according to a control command from the controller 110.

In an embodiment, the controller 110 may analyze a sensing result obtained by the sensing of the sensing unit 120 and control processes to be performed thereafter. For example, the controller 110 may control power to be supplied to the heater 150 to start or end an operation of the heater 150 or the driving unit 190 based on the sensing result obtained by the sensing of the sensing unit 120.

For example, the controller 110 may control an amount of power to be supplied to the heater 150 and a time for which the power is to be supplied, such that the heater 150 may be heated up to a predetermined temperature or maintained at an appropriate temperature, based on the sensing result obtained by the sensing of the sensing unit 120.

In an embodiment, the controller 110 may control the output unit 130 based on the sensing result obtained by the sensing of the sensing unit 120. For example, when the number of puffs counted through the puff sensor 126 reaches a preset number, the controller 110 may inform the user that the inhaler 100 is to end soon through at least one of the display 132, the haptic portion 134, or the sound outputter 136. Alternatively, for example, the puff sensor 126 may sense an inhalation state of a user, and the controller 110 may control driving of the vibration member 191 of the driving unit 190 based on the sensed inhalation state. The controller 110 may adjust an intensity of a vibration of the vibration member 191 based on a value of inhalation pressure sensed by the puff sensor 126. For example, the controller 110 may adjust the intensity of the vibration by adjusting a vibration period and a size of the vibration of the vibration member 191.

In an embodiment, the controller 110 may control a power supply time and/or a power supply amount for the heater 150 according to a state of a stick or a capsule sensed by the sensing unit 120.

An embodiment may also be implemented in the form of a recording medium including instructions executable by a computer, such as a program module executable by the computer. A computer-readable medium may be any available medium that can be accessed by a computer and includes a volatile medium, a non-volatile medium, a removable medium, and a non-removable medium. In addition, the computer-readable medium may include both a computer storage medium and a communication medium. The computer storage medium includes all of a volatile medium, a non-volatile medium, a removable medium, and a non-removable medium implemented by any method or technology for storage of information such as computer-readable instructions, data structures, program modules or other data. The communication medium typically includes a computer-readable command, a data structure, or other data regarding a modulated data signal such as a program module, or other transmission mechanisms, and includes any information transfer medium.

FIG. 2 is a diagram schematically illustrating an inhaler 200 according to an embodiment.

Referring to FIG. 2, the inhaler 200 according to an embodiment may include a holder 210 and the stick 230, but embodiments are not limited thereto. For example, the inhaler 200 may refer to the holder 210, excluding the stick 230. The stick 230 may be formed integrally with the holder 210. Alternatively, the stick 230 (e.g., a cigarette) may be detachable from the holder 210. Hereinafter, the term “inhaler” is interchangeably used with the “holder.”

In an embodiment, the holder 210 may have a cylindrical shape or a polygonal column shape. An insertion groove 215 into which the stick 230 is to be inserted may be formed in the holder 210, and the stick 230 may be inserted into the insertion groove 215 in a first direction (e.g., the −Y direction).

In an embodiment, the holder 210 may include a first surface 211, a second surface 212, and a side surface 213. The insertion groove 215 may be formed in the first surface 211, and the second surface 212 may be a surface opposite to the first surface 211. The side surface 213 may be formed between the first surface 211 and the second surface 212.

In an embodiment, the insertion groove 215 may be configured as a recess formed concavely in a direction from the first surface 211 to the second surface 212 and may be formed to be open toward at least a partial area of the first surface 211.

For example, the insertion groove 215 may have a shape extending in a longitudinal direction (e.g., the Y-axis direction) of the holder 210. The stick 230 may be inserted into the holder 210 in a direction (e.g., the −Y direction or the first direction) in which the insertion groove 215 extends into the holder 210.

In an embodiment, an inlet (not shown) through which air outside the holder 210 may be introduced into the insertion groove 215 may be formed between an external side of the holder 210 and the insertion groove 215.

Although not shown in the drawings, the holder 210 may accommodate various components of the inhaler 200 therein. For example, the holder 210 may accommodate at least one of a controller (e.g., the controller 110 of FIG. 1), at least one sensor (e.g., the sensing unit 120 of FIG. 1), and a battery (e.g., the battery 140 of FIG. 1).

In an embodiment, the stick 230 may have a cylindrical shape or a polygonal column shape and may have a size and shape that may be inserted into the insertion groove 215 of the holder 210. The stick 230 may accommodate powder P therein.

In an embodiment, a mouthpiece 231 may be provided at one end portion of the stick 230. For example, the mouthpiece 231 may be provided at one end portion opposite to an area (e.g., a chamber 233 of FIG. 3A) of the stick 230 that is inserted into the insertion groove 215. A user may inhale air by applying a negative pressure to the stick 230. For example, the user may inhale the powder P, or air or aerosol containing the powder P while holding the mouthpiece 231 in the mouth.

FIG. 3A is a diagram schematically illustrating the inside of the inhaler 200 according to an embodiment, and FIG. 3B is a diagram schematically illustrating the inside of the inhaler 200 according to an embodiment.

Specifically, FIGS. 3A and 3B are diagrams illustrating the inside of an area A shown in FIG. 2. FIG. 3A illustrates a state in which the stick 230 is being inserted, thus partially inserted, into the insertion groove 215 of the holder 210, and FIG. 3B illustrates a state in which the stick 230 is substantially completely inserted into the insertion groove 215 of the holder 210.

Referring to FIGS. 3A and 3B, the inhaler 200 according to an embodiment may include at least one of a piercing member 220, an elastic member 225, the chamber 233, and a piercing hole 234.

In an embodiment, the stick 230 may include the chamber 233 to accommodate the capsule 232. The chamber 233 may be a partial area of the stick 230 that is inserted into the insertion groove 215. The chamber 233 may be a space for accommodating or storing the capsule 232 or a space for restricting movement of the capsule 232.

In an embodiment, the capsule 232 may contain powder P therein. The powder P may be a tobacco extract in a state of small particles, or may be a functional material or a composition including a pharmacological material such as caffeine, taurine, aspirin, a sedative, a sleeping pill, a bronchodilator, or a vaccine, or a material such as a nicotine-free material or nicotine salts. However, this is only an example, and the powder P in the capsule 232 may be replaced with liquid, gas, or a combination of some thereof.

In an embodiment, the piercing hole 234 may be an opening that opens from the outside of the stick 230 toward the chamber 233. The piercing hole 234 may be formed in a surface of the stick 230 facing the insertion groove 215, desirably in an area facing the piercing member 220. The piercing hole 234 may have a diameter greater than or equal to that of the piercing member 220.

In an embodiment, the stick 230 may include an airflow channel 235 that provides fluid communication from the chamber 233 to a mouthpiece (e.g., the mouthpiece 231 of FIG. 2). The airflow channel 235 may be a flow path through which air containing the powder P flows, and a mesh 236 may be arranged between the airflow channel 235 and the chamber 233.

In an embodiment, the mesh 236 may pass the powder P and air and may restrict a passage of the capsule 232 or other foreign materials. Alternatively, the mesh 236 may filter out some of the powder P or assist in preventing agglomeration of the powder P. For example, a single hole of the mesh 236 may have a diameter of 5 micrometers (μm).

In an embodiment, when the capsule 232 is crushed, at least a portion of the powder P in the capsule 232 may be discharged to the chamber 233. When a user inhales air of the stick 230 through the mouthpiece 231, the powder P may pass through the mesh 236, pass through the airflow channel 235, move to the mouthpiece 231, and be inhaled by the user.

In an embodiment, the stick 230 may be disposable and be replaced with another stick 230 when the powder P is completely used. Alternatively, the stick 230 may be used multiple times. When the powder P is completely used, the stick 230 may be refilled with the capsule 232 or the powder P and used again.

In an embodiment, the piercing member 220 may be provided in the insertion groove 215 and may protrude from the insertion groove 215 in a direction (e.g., the +Y direction) toward the stick 230. The piercing member 220 may crush the capsule 232. For example, when the stick 230 is inserted into the insertion groove 215 in the first direction (e.g., the −Y direction), at least a partial area of the piercing member 220 may be inserted into the chamber 233 of the stick 230 by passing through the piercing hole 234 and the piercing member 220 may partially crush the capsule 232.

In an embodiment, a distal end portion of the piercing member 220 may have a sharp or pointed shape. For example, the piercing member 220 may be a needle or a sting. The distal end portion of the piercing member 220 may form a perforation in the capsule 232 by crushing a partial area of the capsule 232. The capsule 232 may discharge the powder P to the chamber 233 through the perforation formed by the piercing member 220.

The elastic member 225 according to an embodiment may be provided in the insertion groove 215. When the stick 230 is inserted into the insertion groove 215, the elastic member 225 may be pressed by the stick 230 to be deformed (e.g., compressed). When the elastic member 225 is deformed, the elastic member 225 may press the stick 230 in a direction (e.g., the +Y direction) opposite to the first direction due to an elastic force. The elastic member 225 may include a coil spring that may apply the elastic force.

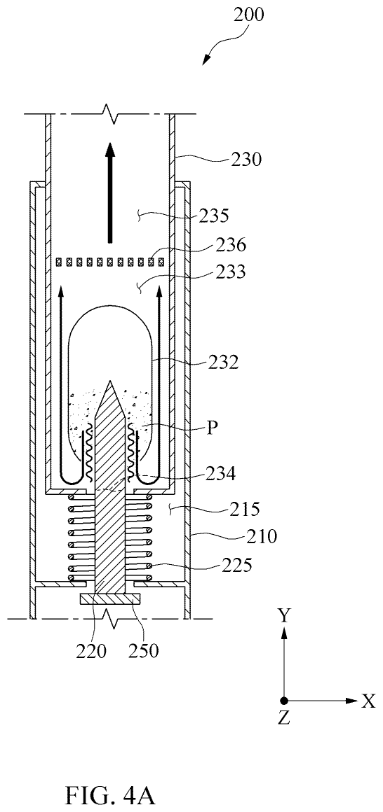

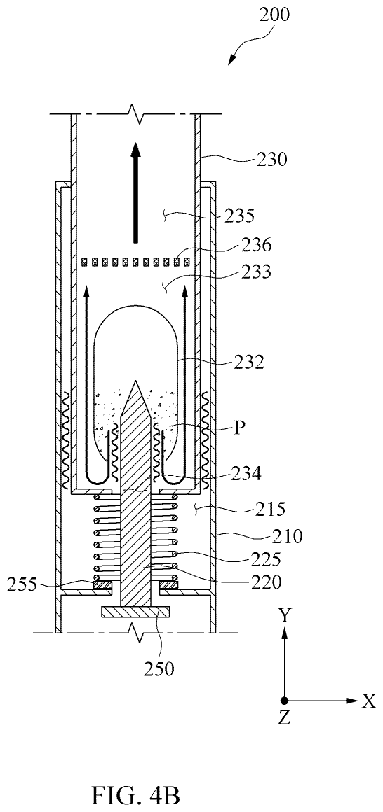

FIG. 4A is a diagram schematically illustrating the inside of the inhaler 200 according to an embodiment.

Referring to FIG. 4A, the inhaler 200 according to an embodiment may include a vibration member 250 (e.g., the vibration member 191 of FIG. 1).

In an embodiment, when the capsule 232 is crushed by the piercing member 220, a perforation communicating with the chamber 233 may be formed in the capsule 232, and at least a portion of the powder P in the capsule 232 may be discharged to the chamber 233 through the perforation. The powder P discharged to the chamber 233 may pass through the mesh 236 and be transferred to the airflow channel 235. After passing through the airflow channel 235, the powder P may pass through a mouthpiece (e.g., the mouthpiece 231 of FIG. 2) and be inhaled by a user.

In an embodiment, a factor, such as an amount of powder P that may be inhaled by a user, may change based on various parameters (e.g., factors such as an amount of powder P discharged per unit time, a density of powder P in the air passing through the airflow channel 235, or a degree to which the discharged powder P spreads) associated with the powder P discharged from the capsule 232.

In an embodiment, by controlling an amount (hereinafter, referred to as a “discharge amount of powder P”) of powder P discharged from the capsule 232 per unit time using the vibration member 250, the inhaler 200 may provide the powder P to a user in accordance with a condition of the user, a use environment, or a preference of the user.

In an embodiment, the vibration member 250 may provide a vibration to the piercing member 220. Alternatively, the vibration member 250 may directly or indirectly vibrate at least one of the capsule 232, the powder P, or the chamber 233. Hereinafter, for convenience of description, an example of the inhaler 200 is described with reference to the drawings in which the vibration member 250 vibrates the piercing member 220.

In an embodiment, the vibration member 250 may be implemented as an electronic vibrator that generates a vibration when a voltage (e.g., AC voltage) is applied to the electronic vibrator. However, embodiments are not limited thereto in actual implementation, and the vibration member 250 may be implemented with various structures and configurations that may provide vibration to the piercing member 220.

In an embodiment, the vibration member 250 may apply a vibration to the piercing member 220 to assist in discharging the powder P in the capsule 232. When the vibration member 250 vibrates the piercing member 220, the perforation formed in the capsule 232 may increase in size, or the vibration may be transmitted to the capsule 232 or the powder P, and thus, the discharge amount of powder P may increase.

In an embodiment, the vibration member 250 may vibrate the piercing member 220 in a direction (e.g., the +/−Y direction) substantially parallel to the first direction (e.g., the −Y direction), that is, a direction in which the stick 230 is inserted into the holder 210.

In an embodiment, when the vibration member 250 vibrates in the direction substantially parallel to the first direction, the powder P that is placed or stagnant between the capsule 232 and the piercing member 220 may be effectively discharged. In addition, the powder P may be prevented from being discharged from the stick 230 through the piercing hole 234, because there is no need for a large diameter of the piercing hole 234 to secure a space for a vibration of the piercing member 220. Also, the vibration of the vibration member 250 may be transferred mainly to the piercing member 220 and the capsule 232, and vibration transferred to the stick 230 or the holder 210 may be reduced or prevented.

In an embodiment, the vibration member 250 may vibrate the piercing member 220 in a direction (e.g., an X-Z plane direction) substantially perpendicular to the first direction (e.g., the −Y direction) in which the stick 230 is inserted into the holder 210. The vibration of the piercing member 220 may be transferred to the capsule 232, and the discharging of the powder P may be promoted due to vibrating of the capsule 232.

In an embodiment in which the vibration member 250 vibrates in the direction substantially perpendicular to the first direction, the piercing member 220 may increase the size of the perforation, or the piercing member 220 may secure a space between the capsule 232 and the piercing member 220 so that the powder P may be more effectively discharged. In addition, the capsule 232 may repeatedly collide with the chamber 233 due to the vibration, and accordingly, the discharge amount of powder P may further increase.

In an embodiment, the vibration member 250 may vibrate the piercing member 220 in a direction substantially parallel to the first direction and in a direction substantially perpendicular to the first direction. The vibration member 250 may three-dimensionally vibrate the capsule 232 through the piercing member 220, to relatively increase the discharge amount of powder P in comparison to a simple one-direction reciprocating motion.

In various embodiments, the inhaler 200 may need to reduce or increase an amount of powder P to be discharged from the capsule 232 or a speed at which the powder P is discharged from the capsule 232 according to various factors including a respiration volume of a user or according to a preference of the user. If this is to be controlled solely by a fixed size of a perforation, it may be difficult to meet demands of various users and various environments.

For example, when the perforation is large, a large amount of the powder P may be discharged within a short period of time, and a density of powder P inhaled by a user may be irregular or abruptly increase. On the other hand, when the perforation is small, it may be difficult to discharge the powder P, and thus, a user with a low lung capacity may have a difficulty in inhaling the powder P. According to an embodiment, the vibration member 250 may control the powder P to be efficiently discharged from the capsule 232 by providing vibration to the capsule 232.

In the inhaler 200 according to various embodiments of the present disclosure, a perforation with a relatively small size may be formed in the capsule 232 by the piercing member 220, and the amount of powder P to be discharged from the capsule 232 per unit time may be controlled through the vibration member 250, and thus, the density of powder P inhaled by a user may be controlled.

FIG. 4B is a diagram schematically illustrating the inside of the inhaler 200 according to an embodiment.

Referring to FIG. 4B, the inhaler 200 according to an embodiment may include a sub-vibration member 255 (e.g., the vibration member 191 of FIG. 1).

In the description of FIG. 4B, description of the inhaler 200 that overlaps with the above description is omitted.

In an embodiment, the sub-vibration member 255 may provide a vibration to the chamber 233. The sub-vibration member 255 may directly or indirectly vibrate the chamber 233 to assist the powder P discharged to the chamber 233 in moving to the airflow channel 235. Alternatively, the sub-vibration member 255 may assist the vibration member 250 to promote discharge of the powder P from the capsule 232.

In an embodiment, as shown in FIG. 4B, the sub-vibration member 255 may be connected to the elastic member 225 to provide vibration to the chamber 233 through the elastic member 225. However, embodiments are not limited thereto, and the sub-vibration member 255 may be implemented with various structures to transfer vibration to the chamber 233. For example, the sub-vibration member 255 may be connected directly to the stick 230, or may be disposed in the holder 210 to apply an impact to the stick 230.

In an embodiment, the chamber 233 may provide kinetic energy to the powder P in the chamber 233 due to the vibration. The powder P may be evenly dispersed due to the vibration of the chamber 233 and may move along airflow. Also, the vibration of the chamber 233 may be transferred to the capsule 232. The sub-vibration member 255 may promote discharge of the powder P by vibrating the capsule 232.

In an embodiment, the sub-vibration member 255 may vibrate the chamber 233 in the direction (e.g., the +/−Y direction) substantially parallel to the first direction (e.g., the −Y direction), that is, the direction in which the stick 230 is inserted into the holder 210. Here, the elastic member 225 may assist and strengthen the vibration of the sub-vibration member 255. In this case, as the chamber 233 vibrates in the direction substantially parallel to the first direction, powder P remaining on the bottom surface of the chamber 233 may be effectively moved, and there may be no need to increase an opening of the insertion groove 215 for the vibration of the chamber 233.

In an embodiment, the sub-vibration member 255 may vibrate the chamber 233 in the direction (e.g., an X-Z plane direction) substantially perpendicular to the first direction in which the stick 230 is inserted into the holder 210. Here, the chamber 233 may repeatedly hit the capsule 232 and thus effectively induce the discharge of the powder P. Also, as the chamber 233 vibrates in the direction substantially perpendicular to the first direction, the powder P may be evenly dispersed in a substantially horizontal direction of the chamber 233.

In an embodiment, the sub-vibration member 255 may vibrate the chamber 233 in a direction substantially parallel to the first direction and in a direction substantially perpendicular to the first direction. The sub-vibration member 255 may three-dimensionally vibrate the capsule 232, to relatively increase the discharge amount of powder P in comparison to a simple one-direction reciprocating motion.

In an embodiment, a processor (e.g., the controller 110 of FIG. 1) may acquire information about driving of the inhaler 200 from various types of sensors (e.g., the sensing unit 120 of FIG. 1), may change various factors, such as the number of vibrations, a vibration intensity, and a vibration time of the vibration member 250 and/or the sub-vibration member 255 based on the acquired information, and may control vibration of each of the vibration member 250 and the sub-vibration member 255.

For example, the controller 110 may receive information about airflow in the stick 230 through a puff sensor (e.g., the puff sensor 126 of FIG. 1). If it is determined that the airflow is insufficient, the controller 110 may increase the discharge amount of powder P by increasing the number of vibrations or vibration intensity of the vibration member 250 and/or the sub-vibration member 255.

Also, the controller 110 may receive a separate input signal from a user input unit (e.g., the user input unit 160 of FIG. 1) or a communication unit (e.g., the communication unit 180 of FIG. 1) and may control vibration of the vibration member 250 and/or the sub-vibration member 255 based on the received signal.

For example, the controller 110 may control the vibration of the vibration member 250 and/or the sub-vibration member 255 according to various factors, such as a respiration volume of a user, a preference of the user, or a use environment. The inhaler 200 according to various embodiments of the present disclosure may help a user smoothly inhale the powder P and may be customized to the user.

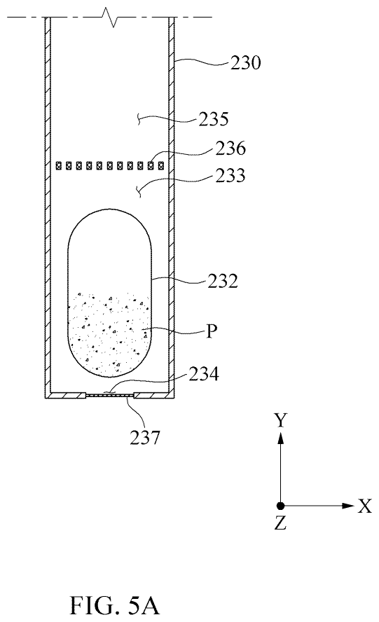

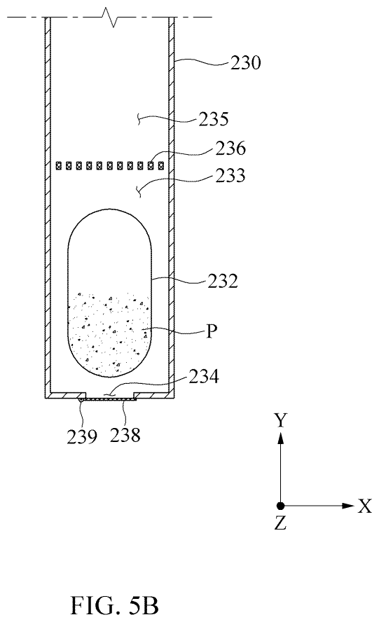

FIG. 5A is a cross-sectional view of the stick 230 according to an embodiment, and FIG. 5B is a cross-sectional view of the stick 230 according to another embodiment. Specifically, FIGS. 5A and 5B are cross-sectional views of a partial area of the stick 230 separated from the holder 210.

Referring to FIGS. 5A and 5B, the stick 230 may include at least one of a sealing member 237 or a door 238.

As shown in FIG. 5A, the sealing member 237 may seal the piercing hole 234. The sealing member 237 may be crushed by the piercing member 220 while the stick 230 is being inserted into the insertion groove 215. For example, the stick 230 may be disposable. Alternatively, the stick 230 may be used multiple times. When the sealing member 237 and the capsule 232 are crushed, the stick 230 may be reused by replacing the sealing member 237 and the capsule 232.

In an embodiment, the sealing member 237 may protect the chamber 233 to prevent water or foreign materials from flowing into the chamber 233 during manufacturing and transportation of the stick 230. Also, the sealing member 237 may limit an area of the piercing hole 234 crushed by the piercing member 220 to prevent the powder P from being discharged from the stick 230 through the piercing hole 234.

As shown in FIG. 5B, the door 238 may selectively open and close the piercing hole 234. The door 238 may be opened before or while the stick 230 is inserted into the insertion groove 215. The door 238 may be moved by a door hinge 239.

For example, the door hinge 239 may push and move the door 238 in a substantially horizontal direction (e.g., the X-Z plane direction), or tilt the door 238 in a substantially vertical direction (e.g., the +/−Y direction).

In an embodiment, the door 238 may be opened when the inhaler 200 is in use or ready for use, and may be closed when the inhaler 200 is not in use. The door 238 may protect the chamber 233 to prevent water or foreign materials from flowing into the chamber 233 during the manufacturing and transportation of the stick 230. Also, the capsule 232 of the chamber 233 which is used or crushed may be replaced through the door 238.

In an embodiment, a processor (e.g., the controller 110 of FIG. 1) may acquire information about coupling of the stick 230 from various types of sensors (e.g., the sensing unit 120 of FIG. 1), and may control driving of the door hinge 239 based on the acquired information.

For example, the controller 110 may detect whether the stick 230 is inserted into the insertion groove 215 or is being inserted, using an insertion detection sensor (e.g., the insertion detection sensor 124 of FIG. 1), and may open the door 238 by controlling the door hinge 239 so that the piercing member 220 may pass through the piercing hole 234. However, embodiments are not limited thereto. The door 238 may be manually opened and closed by a user. For example, a user may manually open and close the door 238, or the inhaler 200 may include a separate switch (not shown) connected to the door 238 so that the user may manipulate the switch to open or close the door 238.

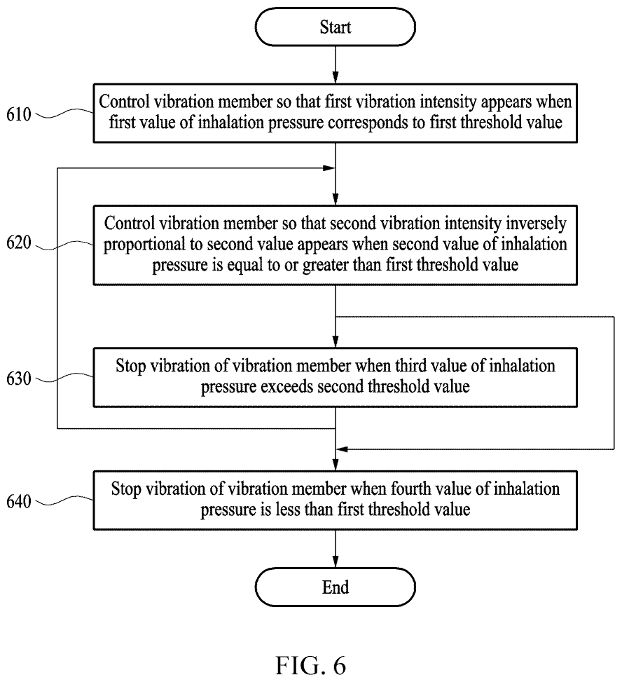

FIG. 6 is a flowchart illustrating a method of controlling a vibration of a vibration member, according to an embodiment.

Operations 610 to 640 may be performed through an inhaler (e.g., the inhaler 100 of FIG. 1 or the inhaler 200 of FIG. 2).

In operation 610, a controller (e.g., the controller 110 of FIG. 1) of the inhaler may vibrate the vibration member (e.g., the vibration member 191 of FIG. 1 or the vibration member 250 of FIG. 4A) so that a first vibration intensity appears when a first value of the inhalation pressure corresponds to a preset first threshold value. For example, the first value of the inhalation pressure may be a value measured by a puff sensor (e.g., the puff sensor 126 of FIG. 1). When a user inhales air by applying a negative pressure to a stick (e.g., the stick 230 of FIG. 2), airflow may be generated in the stick and the puff sensor may sense the generated airflow. The puff sensor may measure the first value of the inhalation pressure by the intensity of the negative pressure. The puff sensor may consecutively measure inhalation pressure values, and in the present disclosure, expressions such as a “first value,” a “second value,” a “third value,” and a “fourth value” are hereinafter used in order to distinguish the inhalation pressure values from each other.

The controller may not vibrate the vibration member from the time when the user starts to apply a negative pressure to the stick until the value of the inhalation pressure reaches the preset first threshold value. When the value of the inhalation pressure increases and reaches (or corresponds to) the preset first threshold value, the controller may vibrate the vibration member so that the first vibration intensity appears. For example, the first vibration intensity may be the maximum vibration intensity. The first vibration intensity may be determined in association with the first threshold value. Since the vibration of the vibration member is provided to assist the user in inhaling the powder, a stronger vibration may be output when the inhalation pressure is weaker.

According to an embodiment, the first threshold value and the first vibration intensity may be personalized for the user. For example, the user may adjust each of the first threshold value and the first vibration intensity through the inhaler or a user terminal communicating with the inhaler. The inhaler may provide a test inhalation mode for the user. The test inhalation mode may be a mode for measuring a change (or trajectory) of the inhalation pressure of the user and suggesting (or adjusting) the first threshold value and the first vibration intensity to the user based on a measured change in the inhalation pressure. The first threshold value and the first vibration intensity may each be set to provide an appropriate powder inhalation amount to the user. For example, as the user's total inhalation time detected through the test inhalation mode increases, the intensity of the vibration for the total inhalation time may generally reduce.

In operation 620, the controller of the inhaler may control the vibration member so that a second vibration intensity inversely proportional to a second value of the inhalation pressure appears when the second value is equal to or greater than the first threshold value. For example, as the value of the inhalation pressure that is equal to or greater than the first threshold value gradually increases, the vibration intensity to appear may gradually decrease. The vibration intensity gradually decreasing as the value of the inhalation pressure gradually increases may be to provide a constant (or appropriate) powder inhalation amount to the user.

According to an embodiment, the vibration intensities may be preset to linearly decrease according to values of the inhalation pressure linearly increasing.

According to an embodiment, the vibration intensities may be preset to non-linearly decrease according to values of the inhalation pressure linearly increasing.

In operation 630, the controller of the inhaler may stop the vibration of the vibration member when a third value of the inhalation pressure exceeds a preset second threshold value. The vibration of the vibration member being stopped when the value of the inhalation pressure is equal to or greater than a preset value may be because an appropriate supply amount is satisfied only with the amount of powder inhaled by the negative pressure of the user and thus there is no need to supply additional powder to the user through the vibration.

According to an embodiment, when the value of the inhalation pressure is equal to or greater than the first threshold value and does not exceed the second threshold value, the vibration intensity of the vibration member corresponding to the value of the inhalation pressure may continuously appear. In other words, even after operation 620 is performed, operation 630 may not be performed according to the value of the inhalation pressure.

According to an embodiment, when the value of the inhalation pressure at first exceeds the second threshold value and then decreases to be equal to or less than the second threshold value, the vibration intensity of the vibration member corresponding to the reduced value of the inhalation pressure may continuously appear. In other words, even after operation 630 is performed, operation 620 may be performed according to the value of the inhalation pressure.

In operation 640, the controller of the inhaler may stop the vibration of the vibration member when a fourth value of the inhalation pressure is less than the first threshold value. For example, when the user performs an operation to complete powder inhalation, the value of the inhalation pressure may indicate a small value. The vibration of the vibration member being stopped when the value of the inhalation pressure is less than a preset value may be because the value of the inhalation pressure means the user has finished inhaling and thus there is no need to supply additional powder to the user through the vibration.

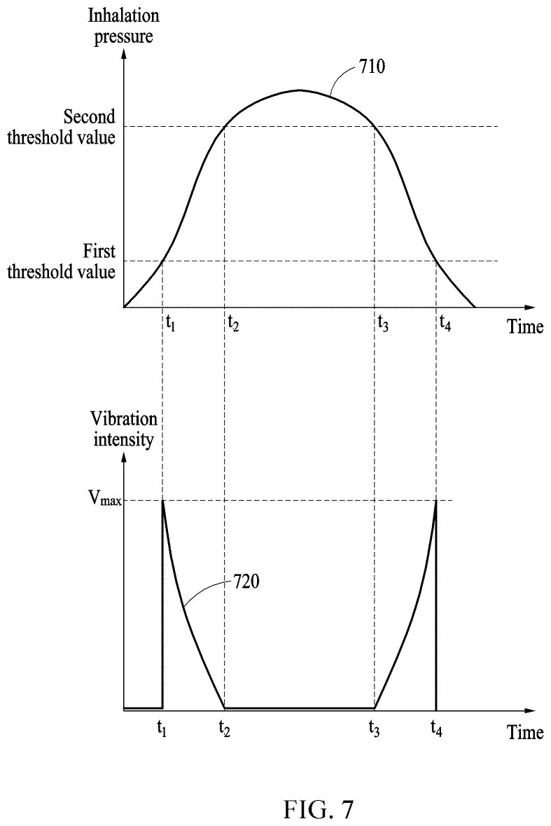

FIG. 7 illustrates a vibration intensity generated according to an inhalation pressure, according to an embodiment.

According to an embodiment, an inhalation pressure change trajectory 710 that appears when a user inhales powder may be illustrated as an axis for time and an axis for an inhalation pressure. For example, the inhalation pressure change trajectory 710 may have a form of non-linearly increasing with time and then starting to non-linearly decrease again at the maximum value. For example, according to the inhalation pressure change trajectory 710, a first threshold value may appear at a time t₁, a second threshold value may appear at a time t₂, the second threshold value may appear at time a t₃, and the first threshold value may appear at a time t₄. The maximum value of the inhalation pressure change trajectory 710 may appear between the time t₂ and the time t_{3.}

According to an embodiment, a vibration intensity trajectory 720 that appears by the vibration member may be illustrated based on the inhalation pressure change trajectory 710. According to the vibration intensity trajectory 720, a vibration may not appear in a section before the time t₁, which is a section in which the value of the inhalation pressure is less than the first threshold value. According to the vibration intensity trajectory 720, the maximum vibration intensity Vmax may appear at the time t₁, which is a time when the value of the inhalation pressure corresponds to the first threshold value, and vibration intensities inversely proportional to increasing values of the inhalation pressure may appear between the time t₁ and the time t₂. According to the vibration intensity trajectory 720, the vibration may be stopped in a section between the time t₂ and the time t₃, which is a section in which the value of the inhalation pressure exceeds the second threshold value. According to the vibration intensity trajectory 720, vibration intensities inversely proportional to decreasing values of the inhalation pressure may appear between a section between the time t₃ and the time t₄, which is a section in which the value of the inhalation pressure is equal to or less than the second threshold value and equal to or greater than the first threshold value. According to the vibration intensity trajectory 720, the maximum vibration intensity Vₘₐₓ may appear at the time t₄, which is the time when the value of the inhalation pressure corresponds to the first threshold value. Although it is shown in FIG. 7 that the maximum vibration intensity Vₘₐₓ that appears at the time t₄ is the same as the maximum vibration intensity Vₘₐₓ that appears at the time t₁, the maximum vibration intensity that appears at the time t₁ and the maximum vibration intensity that appears at the time t₄ may be different from each other depending on an implementation. According to the vibration intensity trajectory 720, vibration may not appear in a section after the time t₄, which is a section in which the value of the inhalation pressure is less than the first threshold value.

While the embodiments are described with reference to a limited number of drawings, it will be apparent to one of ordinary skill in the art that various alterations and modifications in form and details may be made in these embodiments without departing from the spirit and scope of the claims and their equivalents. For example, suitable results may be achieved if the described techniques are performed in a different order and/or if components in a described system, architecture, device, or circuit are combined in a different manner and/or replaced or supplemented by other components or their equivalents. Therefore, other implementations, other embodiments, and/or equivalents of the claims are within the scope of the following claims.

1. An inhaler comprising:
a stick comprising a chamber configured to accommodate a capsule comprising powder and a piercing hole open toward the chamber;
a holder comprising an insertion groove into which the stick is inserted in a first direction;
a piercing member provided in the insertion groove and configured to crush the capsule by passing through the piercing hole when the stick is inserted into the insertion groove;
a vibration member configured to provide a vibration to the piercing member;
a puff sensor configured to sense airflow inside the stick; and
a controller configured to control an operation of the inhaler.

2. The inhaler of claim 1, wherein the controller is configured to adjust an intensity of a vibration of the vibration member based on a value of an inhalation pressure sensed by the puff sensor.

3. The inhaler of claim 2, wherein the controller is configured to control the vibration member so that a preset first vibration intensity appears when the value of the inhalation pressure corresponds to a preset first threshold value.

4. The inhaler of claim 3, wherein the controller is configured to stop the vibration of the vibration member when the value of the inhalation pressure exceeds a preset second threshold value.

5. The inhaler of claim 4, wherein the controller is configured to control the vibration member so that a vibration intensity inversely proportional to the value of the inhalation pressure appears when the value of the inhalation pressure is greater than the preset first threshold value and equal to or less than the preset second threshold value.

6. The inhaler of claim 3, wherein the preset first threshold value is a personalized value for a user of the inhaler.

7. The inhaler of claim 1, further comprising:
a sub-vibration member configured to provide a vibration to the chamber.

8. The inhaler of claim 7, further comprising:
an elastic member provided in the insertion groove and pressed by the stick when the stick is inserted,
wherein the sub-vibration member is configured to provide the vibration to the chamber of the stick through the elastic member.

9. The inhaler of claim 7, wherein the sub-vibration member is configured to vibrate the chamber in a direction substantially parallel to the first direction.

10. The inhaler of claim 7, the sub-vibration member is configured to vibrate the chamber in a direction substantially perpendicular to the first direction.

11. The inhaler of claim 1, wherein the stick further comprises:
a mouthpiece provided on an opposite side of the chamber;
an airflow channel configured to communicate from the chamber to the mouthpiece; and
a mesh arranged between the airflow channel and the chamber.

12. The inhaler of claim 1, wherein the stick further comprises:
a sealing member configured to seal the piercing hole and crushed by the piercing member when the stick is inserted into the insertion groove.

13. A method of providing a vibration for powder inhalation, the method being performed by an inhaler, wherein the inhaler comprises:
a stick comprising a chamber configured to accommodate a capsule comprising powder and a piercing hole open toward the chamber;
a holder comprising an insertion groove into which the stick is inserted in a first direction;
a piercing member provided in the insertion groove and configured to crush the capsule by passing through the piercing hole when the stick is inserted into the insertion groove;
a vibration member configured to provide a vibration to the piercing member;
a puff sensor configured to sense airflow inside the stick; and
a controller configured to control an operation of the inhaler,
wherein the method comprises:
controlling the vibration member so that a preset first vibration intensity appears when a first value of an inhalation pressure measured by the puff sensor corresponds to a preset first threshold value; and
controlling the vibration member so that a second vibration intensity inversely proportional to a second value of the inhalation pressure appears when the second value is equal to or greater than the preset first threshold value.

14. The method of claim 13, further comprising:
stopping the vibration of the vibration member when a third value of the inhalation pressure exceeds a preset second threshold value.

15. The method of claim 13, further comprising:
stopping the vibration of the vibration member when a fourth value of the inhalation pressure is less than the preset first threshold value.
